(19) United States
(12) Patent Application Publication
(10) Pub. No.: US 2007/0106113 A1
(43) Pub. Date: May 10, 2007

(54) COMBINATION ENDOSCOPIC OPERATIVE DELIVERY SYSTEM

(76) Inventor: Biagio Ravo, Rome (IT)

Correspondence Address: 24IP LAW GROUP USA, PLLC, 12 E. LAKE DRIVE, ANNAPOLIS, MD, 21403, US

(21) Appl. No.: 11/556,743

(22) Filed: Nov. 6, 2006

Related U.S. Application Data
(60) Provisional application No. 60/734,182, filed on Nov. 7, 2005.

(51) Int. Cl. A61B 1/00

(52) U.S. Cl. 600/113; 600/114; 600/128; 600/153

(57) ABSTRACT

An endoscope for performing minimally invasive surgery includes along a portion of its length a means such as a groove for slidingly engaging with a corresponding means such as a tongue in a second endoscope and guiding the second endoscope into the patient. Multiple endoscope may be connected together and guide one another into or out of a patient. The tongue structure may include incongruous portions to permit two endoscopes to be partially connected and guided by one another.

### CROSS-REFERENCE TO RELATED APPLICATIONS

The present application claims the benefit of the filing date of U.S. Provisional Application Ser. No. 60/734,182 entitled “Combination Laparoscopic and Endoscopic Delivery System,” and filed on Nov. 7, 2005.

The above cross-referenced related application is hereby incorporated by reference herein in its entirety.

### STATEMENT REGARDING FEDERALLY SPONSORED RESEARCH OR DEVELOPMENT

None.

### BACKGROUND OF THE INVENTION

1. Field of the Invention

The present invention relates to surgical devices for use in endoscopic and minimally invasive surgery.

2. Brief Description of the Related Art

Endoscopic and minimally invasive surgery has been known for many years and recently has become increasingly useful in treating various medical conditions. In such surgery, a class of devices commonly known as endoscopes may be used. The term endoscope is used here in its broad sense, encompassing all kinds of “scopes” such as laparoscope, colonoscopes, etc.

An endoscope typically but not necessarily has an elongated body having one or more channels therein. Various devices, such as snares, biopsy forceps or other surgical tools, may be inserted into the channels in the endoscope or may be built into, mounted into, or on the endoscope. Thus, once the endoscope is placed into the patient, a surgeon may use a variety of surgical tools by placing them into or removing them form the endoscope. An endoscope may have one or a plurality of channels therein, and the channels may be of a variety of sizes and shapes and are limited only by size of the endoscope.

Traditional endoscopes have suffered from a shortcoming in that once an endoscope has been chosen for a particular surgery and has been inserted into a patient, the surgeon is limited to tools that fit into the number and types of channels in that particular endoscope. Often, the surgeon needs to remove the endoscope during the procedure, such as to remove a specimen, and then re-insert the endoscope or another device. Thus, if a surgeon desired to use a variety of different surgical instruments or tools (or optics), the surgeon often must remove one tool or the like to make available a channel in the endoscope for another tool.

### SUMMARY OF THE INVENTION

The present invention is a flexible or rigid endoscope or channel, or plurality of endoscopes or channels, having a mechanism for permitting the attachment of a second endoscope or channel of various shapes or sizes to the outside of the endoscope via a sliding engagement mechanism such as a tongue and groove structure such that when a first endoscope is within a patient, a surgeon may insert a second endoscope into the patient and have the second endoscope in sliding engagement with the first endoscope such that the first endoscope guides the insertion of the second endoscope into the patient. The channels or endoscopes may be controlled by manual or robotic means.

In an embodiment of the invention, an endoscope comprises an elongated body; at least one operative channel in the body; an engagement means, located along a circumference of said body, for engaging with a second endoscope or other operative channel and guiding the second endoscope or other operative channel into a patient. The engagement means may comprise a groove or tongue or other sliding engagement means parallel to the length of the body of the endoscope.

Another embodiment of the invention comprises first and second endoscopes, each having a body and at least one channel therein, wherein the first endoscope further comprises a groove parallel to a length of its body; and the second endoscope further comprises a tongue parallel to a length of its body for engaging with said groove of the first endoscope. The first or second endoscope may further comprise second, third, or fourth engagement means parallel to their length for engaging with additional endoscope or surgical tools.

In still other embodiments, an endoscope of the present invention has an engagement member that comprises a magnetic strip. An endoscope in accordance with the present invention further may comprise a plurality of ridges, grooves, or a combination of ridges and grooves spaced along a length of said endoscope.

In another embodiment, the present invention is an endoscope that comprises an elongated body having an inner periphery and an outer periphery and an engagement means, member or mechanism on the inner periphery of the body for engaging with a complementary engagement means on a different item and for guiding that item into the body. The item may comprise a sheath having a channel therein for inserting surgical instruments or may comprise other endoscopes, optical means, surgical tools, probes or instruments.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a are perspective view of a dual endoscope in accordance with a preferred embodiment of the invention in a disconnected position.

FIGS. 2a and b are end views of a dual endoscope in connected and disconnected positions in accordance with a preferred embodiment of the present invention.

FIG. 3 is a perspective view of a triple endoscope in accordance with a preferred embodiment of the invention in a connected position.

FIG. 4 is an end view of a triple endoscope in accordance with a preferred embodiment of the present invention in a disconnected position.

FIGS. 5a and b are end view of alternative embodiments of endoscopes in accordance with preferred embodiments of the present invention.

FIG. 6 is a side view of an endoscope having an alternative embodiment of an engagement means in accordance with the present invention.

FIG. 7 is a perspective view of a dual endoscope incorporating an alternative embodiment of the engagement means in accordance with the present invention.

FIG. 8 is an end view of alternative embodiment of an endoscope having moveable or removable sheaths or channels within the endoscope.

### DETAILED DESCRIPTION OF THE PREFERRED EMBODIMENTS

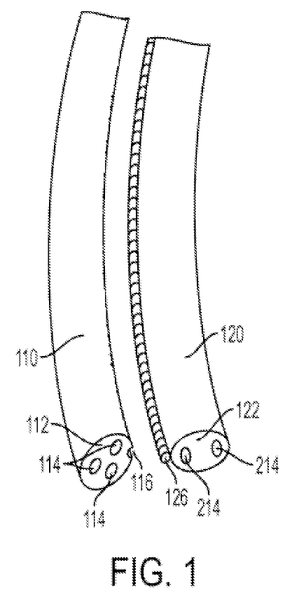

A first preferred embodiment of the invention is shown in FIGS. 1a and b and 2a and b. A first endoscope 110 has one or more channels 114 therein with ports at a distal end 112 of the endoscope 110. While the endoscope 110 shown in FIGS. 1a and b is a flexible endoscope, the present invention may be used with rigid or flexible endoscopes of any type. The channel(s) 114 may be of any type and for any use, whether they be for inserting optics or surgical tools or for suction or delivery of fluid, plasma, gas or the like. The endoscope 110 further has a means located along at least a portion of its length for attaching and/or guiding another device. Such “another device” may be a second endoscope 120 having a distal end 122 and one or more channels 124 having ports at the distal end 122 of the endoscope 120. Like the channel(s) 114, the channels 124 may be of any type and for any use. The endoscope 120 has a means 126 for engaging with the means 116 on the endoscope 110. Preferably, the two endoscopes (or devices) are in sliding engagement such that the endoscope that first was inserted into a patient may act as a guide for insertion of the second endoscope or device. The means 116 and 126 may be of any type such as a tongue and groove, rounded ridge and annular groove, or other engagement means such as magnetic strips. The engagement means may be of any size or shape and is not limited a tongue, ridge or groove. Each endoscope may or may not have its own controller (manual or power) at its proximal end.

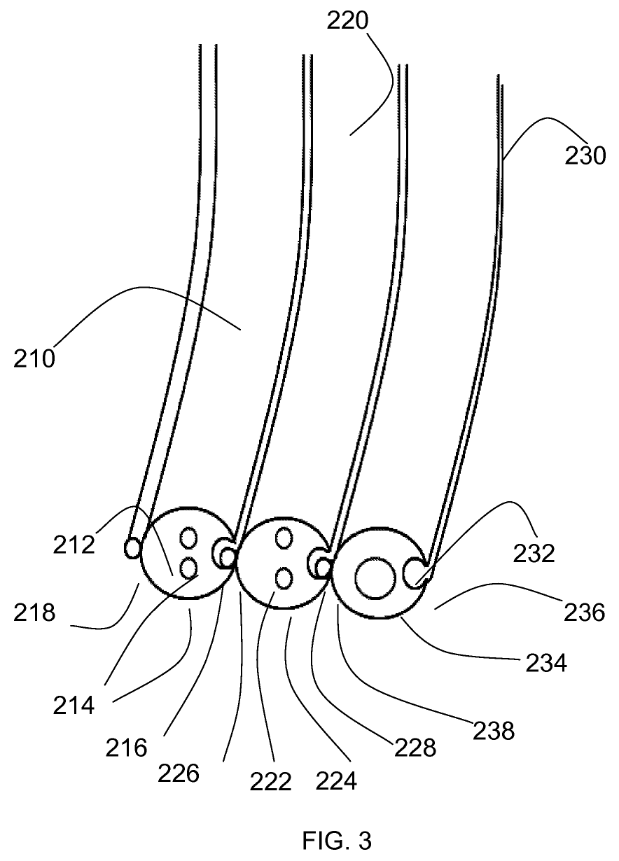

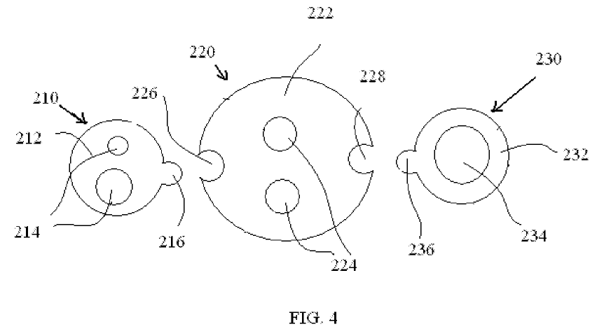

A second preferred embodiment is shown in FIG. 3. In this second preferred embodiment an endoscope 220 has a distal end 222 and one or more channels 224 therein. The endoscope 220 has means 226 and 228 at different positions around its circumference for attaching and/or guiding additional endoscopes or other devices along its length. In FIG. 3, engagement or guiding means 226 is shown as protruding from endoscope 220 and engagement or guiding means 228 is shown as being concave. As shown in FIG. 4, variation on this arrangement are possible such that both engagement means on endoscope are concave. Alternatively, the both may be convex. Returning to FIG. 3, a second endoscope 210 is shown in engagement with the endoscope 220. The second endoscope 210 has a distal end 212 and a plurality of channels 214 therein. The endoscope 210 further has a means 216 for engaging with the means 226 in endoscope 220, such that one of the endoscopes 210, 220 may act as a guide for the other during insertion into the patient. An engagement means 218 is shown on an opposite side of endoscope 210. A third endoscope 230 is shown in FIG. 3. The third endoscope 230 has a distal end 232 and a channel 234 therein. The third endoscope 230 further has a means 238 for engaging with the means 228 in endoscope 220 such that one of endoscopes 220, 230 may act as a guide for the other during insertion into the patient. A concave engagement means 236 is shown on an opposite side of endoscope 230.

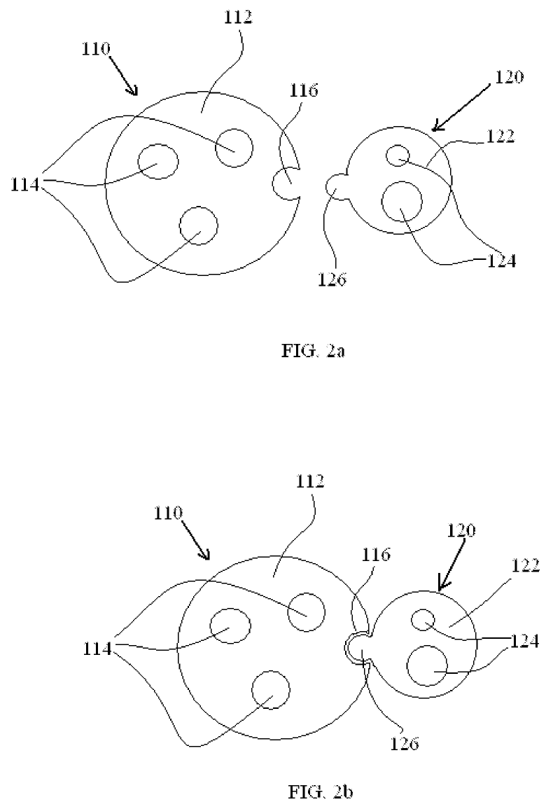

The present invention provides surgeons with great flexibility in performing minimally invasive surgery. The surgeon may use any of a variety of different endoscope having a variety of different channels, tools, delivery mechanisms, suction tubes, etc. therein. Further, while all of the endoscope may have optics therein, the present invention allows for optics to be placed in only one of the endoscope shown in FIGS. 1-4, because the surgeon may view the tools or items inserted into the channels of the second and third endoscope with the optics in the first endoscope. This provides further flexibility for the surgeon.

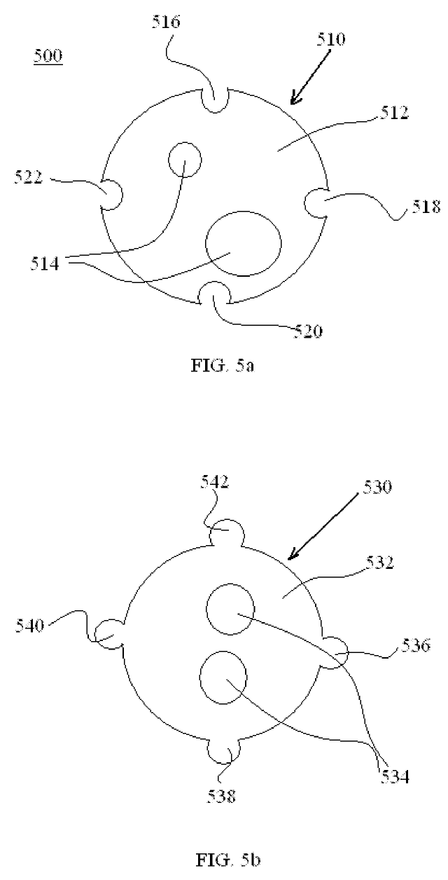

FIGS. 5a and b each show an embodiment of the invention in which an endoscope has engagement means or members for connecting four endoscopes to the endoscope shown. In FIG. 5a, the endoscope has four ridges placed around its circumference. In FIG. 5b, the endoscope is shown as having four grooves around its outer circumference. While four engagement means are shown in FIGS. 5a and 5 are shown, any number of engagement means may be used. Similarly, a single endoscope may have combinations of different types of engagement means around its circumference, such as two grooves and two ridges.

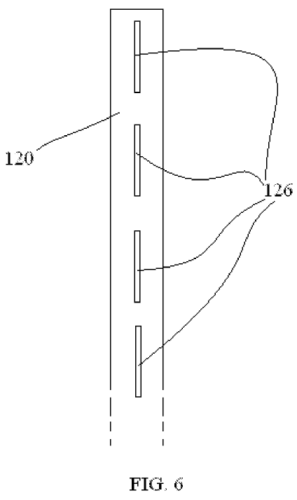

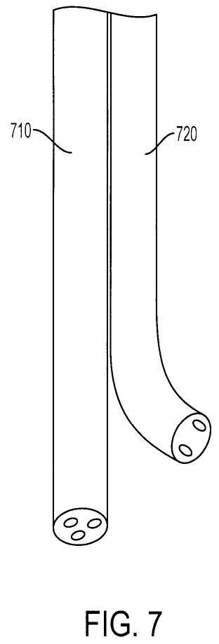

FIG. 6 is a side view of an endoscope in which the engagement means is a series of ridges along its length with reach ridge extending along a portion of the length of the endoscope. With this type of engagement means, a portion of the length of the endoscope may be attached to the length of an endoscope having a complimentary or mating engagement means or mechanism. As shown in FIG. 7, with this type of embodiment, the distal ends of the two connected endoscope may be moved independently of one another.

In each of the disclosed embodiments, the endoscopes being connected to one another may be the same or may be different in type, size or configuration. Likewise, each may or may not have its own optical means or may or may not have suction means.

Many variations of the present invention are possible. For example, scopes having different types or numbers of channels may be combined using the present invention. Further, scopes of different types may even be combined if that is desired by the surgeon.

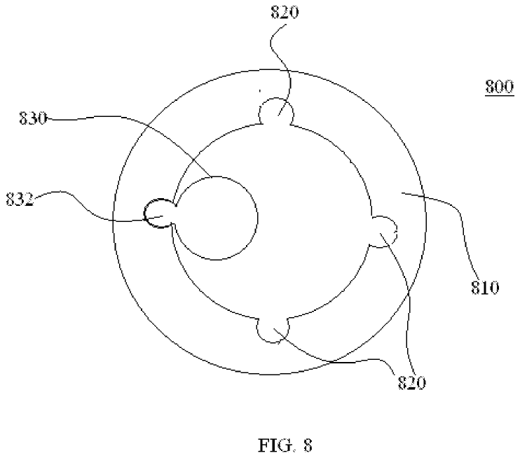

In yet another embodiment shown in FIG. 8, an endoscope 800 has a body 810 having one or more engagement means, mechanism or member 820 along its inner periphery to permit the insertion, removal, movement, and control of one or more channels or sheaths 830 within the endoscope. The channel or sheath 830 has an engagement means or mechanism 832 for engaging with the engagement means or mechanism 820 of the endoscope body 810. Similarly, instruments having corresponding engagement means could be connected along the inner periphery of the endoscope rather than being inserted into a channel or sheath 830. Any engagement mechanism, means or member may be used to connect the sheath or channel to the endoscope. Additionally, the endoscope may have engagement mechanisms, means or members along its outer periphery for the attachment or guidance of additional endoscopes, channels or tools.

With the present invention, a plurality of endoscopes may be connected together in a manner in which one endoscope may be used to guide a second endoscope into a patient. The second endoscope may be inserted to the same depth into the patient as the first endoscope, less depth into the patient or greater depth into the patient. Similarly, rather than a plurality of endoscope being connected together or one endoscope guiding other endoscopes into a patient, the present invention permits tools or instruments to be connected to a first endoscope and be guided into a patient.

Additionally, in each embodiment of the present invention, a locking means, mechanism or device may be used to hold the various endoscopes and tools in desired positions relative to each other. The locking means may be part of one or more of the endoscopes or may be a separate component that is attached to the endoscope(s) once they are in their desired positions relative to each other. The locking mechanism or means may take any form such as a clamp, band or other means.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. An endoscope comprising:
an elongated body;
at least one channel in said body;
engagement means, located along at least a portion of a length of said body, for engaging with a second endoscope and guiding said second endoscope into a patient.

2. An endoscope according to claim 1 further comprising a second engagement means located along at least a portion of a length of said body and a circumferential position on said body different than a circumferential position of said first engagement means on said body.

3. An endoscope according to claim 1 wherein said engagement means comprises a groove along at least a portion of a length of said body

4. An endoscope according to claim 1 wherein said engagement means comprises a tongue along at least a portion of a length of said body.

5. An apparatus comprising first and second endoscopes, each having a body and at least one channel therein, wherein:
said first endoscope further comprises a groove parallel to at least a partial length of its body; and
said second endoscope further comprises a tongue parallel to at least a partial length of its body for engaging with said groove of said first endoscope.

6. An apparatus according to claim 5, wherein said first endoscope further comprises a second groove parallel to at least a partial length of its body at a different radial position on its circumference.

7. An apparatus according to claim 5, wherein said first endoscope further comprises a tongue parallel to at least a partial length of its body at a radial position on its circumference different that said groove.

8. An apparatus according to claim 5, wherein said second endoscope further comprises a second tongue parallel to at least a partial length of its body at a different radial position on its circumference.

9. An endoscope comprising:
an elongated body;
at least one channel in said body;
engagement member, located along at least a portion of a length of said body, for engaging with a second component and guiding said second component into a patient.

10. An endoscope according to claim 9 wherein said engagement member is located on an outer periphery of said endoscope.

11. An endoscope according to claim 10 wherein said endoscope has a plurality of engagement members spaced circumferentially around is outer periphery.

12. An endoscope according to claim 9 wherein said engagement member is located on an inner periphery of said endoscope.

13. An endoscope according to claim 12 wherein said endoscope has a plurality of engagement members spaced radially around is inner periphery.

14. An endoscope according to claim 9, wherein said endoscope has a plurality of engagement members.

15. An endoscope according to claim 9 wherein said engagement member comprises a ridge.

16. An endoscope according to claim 9 wherein said engagement member comprises a magnetic strip.

17. An endoscope according to claim 9 wherein said engagement member comprises a plurality of ridges spaced along a length of said endoscope.

18. An endoscope according to claim 9 wherein said engagement member comprises a groove.

19. An endoscope comprising:
a elongated body having an inner periphery and an outer periphery; and
an engagement means on said inner periphery of said body for engaging with a complementary engagement means on a different item and for guiding said item into said body.

20. An endoscope according to claim 19, wherein said item comprises a sheath having a channel therein for inserting surgical instruments.
